(19) United States
(12) Patent Application Publication
(10) Pub. No.: US 2026/0294551 A1
(43) Pub. Date: Oct. 1, 2026

(54) HARDWARE-BASED TARGET LOCALIZATION FOR PERCUTANEOUS ACCESS

(71) Applicant: Auris Health, Inc., Santa Clara, CA (US)

(72) Inventors: Lauren Elizabeth Friend, San Francisco, CA (US); Jacob William Caldwell, San Francisco, CA (US); William Lanphier Chapin, Redwood City, CA (US); Justin Gilbert, Santa Clara, CA (US)

(73) Assignee: Auris Health, Inc., Santa Clara, CA (US)

(21) Appl. No.: 19/630,275

(22) Filed: Mar. 26, 2026

Related U.S. Application Data
(60) Provisional application No. 63/778,832, filed on Mar. 27, 2025.

(51) Int. Cl. A61B 34/20; A61B 1/00; A61B 1/018; A61B 1/307; A61B 34/30

(52) U.S. Cl. CPC A61B 34/20; A61B 1/00147; A61B 1/018; A61B 1/307; A61B 34/30; A61B 2034/2051; A61B 2034/301

(57) ABSTRACT

This disclosure provides methods, devices, and systems for navigating medical instruments. The present implementations more specifically relate to hardware-based target localization techniques for percutaneous access. In some aspects, one or more sensors may be disposed on an elongate member to form a “working channel instrument” that can be inserted through the working channel of an endoscope. The sensors may implement any sensing technology suitable for tracking the positions of the sensors. The working channel instrument can thus be used to designate a target location within an anatomy. In some implementations, the working channel instrument may include a sensor disposed on the distal tip of a flexible wire. In some other implementations, the working channel instrument may include a series of sensors disposed along a length of flexible wire. Still further, in some implementations, the working channel instrument may include a sensor disposed on a shaft having a barbed tip.

### CROSS-REFERENCE TO RELATED APPLICATIONS

This application claims priority and benefit under 35 U.S.C. § 119(e) to U.S. Provisional Patent Application No. 63/778,832, filed Mar. 27, 2025, which is incorporated herein by reference in its entirety.

### TECHNICAL FIELD

This disclosure relates generally to medical systems, and specifically to hardware-based target localization techniques for percutaneous access.

### DESCRIPTION OF RELATED ART

Many medical procedures, such as laparoscopy, ureteroscopy, or percutaneous nephrolithotomy (PCNL), involve a series of complex steps that require careful movement and positioning of medical tools or instruments inside a patient’s anatomy. For example, to remove urinary stones from the bladder and ureter, a physician can insert a ureteroscope into the urinary tract through the urethra. A ureteroscope includes an endoscope at its distal end configured to enable visualization of the urinary tract. During some percutaneous access procedures, the ureteroscope can be used to designate or set a target location for a needle to access the kidney percutaneously (also referred to as “target selection” or “target localization”). The physician drives the needle into the patient, to the target location, and proceeds to dilate the tract and perform a PCNL procedure. For example, the physician may use another medical instrument (which may be in conjunction with the needle) to extract the stone from the kidney via the percutaneous access point.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method performed by a medical system. The method includes steps of driving a first medical instrument within an anatomy, where the first medical instrument includes a working channel; capturing sensor data from one or more sensors disposed on a distal portion of a second medical instrument that is inserted through the working channel of the first medical instrument, where the sensor data indicates positions of the one or more sensors within the anatomy; and determining a target location within the anatomy for percutaneous access based at least in part on the sensor data.

Another innovative aspect of the subject matter of this disclosure can be implemented in a medical instrument including an elongate member configured to be inserted through a working channel of an endoscope, where the elongate member has a distal portion configured to contact an anatomy; and one or more sensors disposed on the distal portion of the elongate member, where the one or more sensors are configured to produce sensor data indicating positions of the one or more sensors within the anatomy.

Another innovative aspect of the subject matter of this disclosure can be implemented in a controller for a medical system, including a processing system and a memory. The memory stores instructions that, when executed by the processing system, cause the controller to drive a first medical instrument within an anatomy, where the first medical instrument includes a working channel; capture sensor data from one or more sensors disposed on a distal portion of a second medical instrument that is inserted through the working channel of the first medical instrument, where the sensor data indicates positions of the one or more sensors within the anatomy; and determine a target location within the anatomy for percutaneous access based at least in part on the sensor data.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows another example medical system, according to some implementations.

FIGS. 3–5 show an example percutaneous access procedure that can be performed using the medical system of FIG. 1.

FIG. 6 shows a block diagram of an example target localization system, according to some implementations.

FIG. 7 shows an example positioning of a working channel instrument for designating a target for percutaneous access in an anatomy, according to some implementations.

FIG. 8 shows another example positioning of a working channel instrument for designating a target for percutaneous access in an anatomy, according to some implementations.

FIG. 9 shows another example positioning of a working channel instrument for designating a target for percutaneous access in an anatomy, according to some implementations.

FIG. 10 shows an example working channel instrument, according to some implementations.

FIG. 11 shows an example instrument assembly that includes a working channel instrument and an endoscope, according to some implementations.

FIG. 12 shows a block diagram of an example controller for a medical system, according to some implementations.

FIG. 13 shows an illustrative flowchart depicting an example target localization operation, according to some implementations

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system’s registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures involve a series of complex steps that require careful movement and positioning of medical tools or instruments inside an anatomy. For example, to remove urinary stones from the bladder and ureter, a physician can insert a scope (such as a ureteroscope) into the urinary tract through the urethra. A ureteroscope includes an endoscope at its distal end configured to enable visualization of the urinary tract. During some percutaneous access procedures, the ureteroscope can be used to designate or set a target location for a needle to access the kidney percutaneously (also referred to as “target selection” or “target localization”). As used herein, the term “target location” refers to any point or position in a given coordinate frame, which may or may not coincide with a physical object. For example, a target location can be designated within a calyx, midway between a papilla and the distal end of the ureteroscope. The physician drives the needle into the patient, to the target location, and proceeds to dilate the tract and perform a PCNL procedure.

In some medical systems, the distal end of the endoscope is used to designate the target location. For example, an electromagnetic (EM) sensor can be coupled to, embedded in, or otherwise disposed on the distal end of the endoscope. The EM sensor induces current in the presence of an EM field, which can indicate the position of the EM sensor in a coordinate space associated with the EM field. In this way, the distal end of the endoscope can be tracked in the EM coordinate space so that the position of the scope tip can be used as a frame of reference for selecting or designating the target location. However, due to increased manufacturing costs associated with adding EM sensors to an endoscope, many existing endoscopes do not have integrated EM sensors. On the other hand, many existing endoscopes have lumens (also referred to as a “working channel”) in which various other tools can be inserted (such as lithotripters, basket retrieval devices, or forceps).

Aspects of the present disclosure recognize that the working channel of an endoscope can be used to introduce one or more sensors into an anatomy for designating a target location. The sensors may implement any sensing technology (such as EM) suitable for tracking the positions of the sensors (also referred to as “position sensors”) in a given coordinate space. In some aspects, the position sensors may be coupled to or disposed on an elongate member (such as a flexible wire or shaft) to form a “working channel instrument” that can be inserted through the working channel of an endoscope. In some implementations, a working channel instrument may include a position sensor disposed on the distal tip of a flexible wire that can be pressed and/or held in contact with an anatomical surface (such as a papilla). In some other implementations, a working channel instrument may include a series of position sensors disposed along a length of flexible wire that can coil or otherwise deform around an anatomical volume (such as a calyx). Still further, in some implementations, a working channel instrument may include a position sensor disposed on a shaft having a barbed tip that can penetrate and/or latch onto an anatomical surface (such as a papilla).

Particular implementations of the subject matter described in this disclosure can be implemented to realize one or more of the following potential advantages. By leveraging the working channel of an endoscope to introduce position sensors into an anatomy, aspects of the present disclosure can significantly reduce the cost of tools needed to perform a percutaneous access procedure. For example, the working channel instruments of the present disclosure are generally much less complex and thus cheaper to manufacture than articulable endoscopes with integrated sensors. Such working channel instruments can be used in conjunction with existing endoscopes having working channels or lumens, thereby reducing the need for specialized equipment to perform a percutaneous access procedure. Further, because the working channel instruments of the present disclosure can be placed in contact with the anatomy, any changes or movements of the anatomy (such as due to respiration) may result in corresponding changes in the sensor positions. Accordingly, any target location designated based on the positions of such sensors also tracks the movements of the anatomy, which can significantly improve the outcome of a percutaneous access procedure.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient’s anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures that utilize camera and/or sensor data (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples).

Although certain aspects of the present disclosure are described in detail herein in the context of renal, urological, or nephrological procedures, such as kidney stone removal and treatment procedures, it should be understood that such context is provided for convenience and clarity, and the concepts disclosed herein are applicable to any suitable medical procedure. However, as mentioned, description of the renal or urinary anatomy and associated medical issues and procedures is presented herein to aid in the description of the concepts disclosed herein. In some implementations, the techniques and systems described herein are discussed in the context of a percutaneous procedure, which can include any procedure where access is gained to a target location by making a puncture or incision in the skin, mucous membrane, or other body layer. However, it should be understood that these techniques and systems can be implemented in the context of any medical procedure.

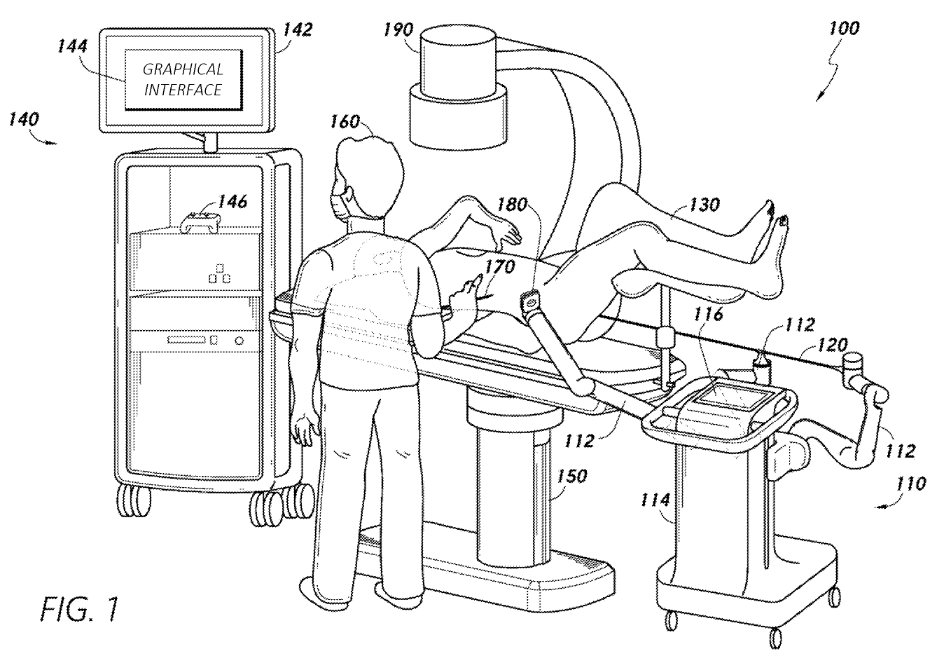

FIG. 1 shows an example medical system 100, according to some implementations. The medical system 100 includes a robotic system 110 configured to engage with and/or control a medical instrument 120 to perform a procedure on a patient 130. The medical system 100 also includes a control system 140 configured to interface with the robotic system 110, provide information regarding the procedure, and/or perform a variety of other operations. For example, the control system 140 can include one or more displays 142 to present certain information to assist the physician 160. The medical system 100 can include a table 150 configured to hold the patient 130. The system 100 further includes an electromagnetic (EM) field generator 180, which can be held by one or more robotic arms 112 of the robotic system 110 or can be a stand-alone device. In the example of FIG. 1, the medical system 100 is shown to include an imaging device 190 which can be integrated into a C-arm or otherwise configured to provide imaging during a procedure, such as for a fluoroscopy-type procedure. In some other implementations, the medical system 100 my not include the imaging device 190.

In some implementations, the medical system 100 may be used to perform a percutaneous access procedure. For example, if the patient 130 has a kidney stone that is too large to be removed through a urinary tract, the physician 160 can perform a procedure to remove the kidney stone through a percutaneous access point on the patient 130. To illustrate, the physician 160 can interact with the control system 140 to control the robotic system 110 to advance and navigate the medical instrument 120 (such as an endoscope) from the urethra, through the bladder, up the ureter, and into the kidney where the stone is located. The control system 140 can provide information via the display(s) 142 regarding the medical instrument 120 to assist the physician 160 in navigating the medical instrument 120, such as real-time images captured therewith.

Once at the site of the kidney stone (such as within a calyx of the kidney), the medical instrument 120 can be used to designate or tag a target location for the medical instrument 170 to access the kidney percutaneously (such as a desired point to access the kidney). To minimize damage to the kidney and/or the surrounding anatomy, the physician 160 can designate a particular papilla as the target location for entering into the kidney with the medical instrument 170. However, other target locations can be designated or determined. To assist the physician in inserting the medical instrument 170 into the patient 130 through the particular papilla, the control system 140 may provide an graphical interface 144, which can include a visualization to indicate an alignment of an orientation of the medical instrument 170 relative to a target trajectory (such as a desired access path), a visualization to indicate a progress of inserting the medical instrument 170 towards the target location, and/or other information. Once the medical instrument 170 has reached the target location, the physician 160 can use the medical instrument 170 and/or another medical instrument to extract the kidney stone from the patient 130, such as through the percutaneous access point.

Although the above percutaneous procedure and/or other procedures are discussed in the context of using the medical instrument 120, in some implementations a percutaneous procedure can be performed without the assistance of the medical instrument 120. Further, the medical system 100 can be used to perform a variety of other procedures. Moreover, although many implementations describe the physician 160 using the medical instrument 170, the medical instrument 170 can alternatively be used by a component of the medical system 100. For example, the medical instrument 170 can be held or manipulated by the robotic system 110 (such as the one or more robotic arms 112) and the techniques discussed herein can be implemented to control the robotic system 110 to insert the medical instrument 170 with the appropriate orientation to reach a target location.

In the example of FIG. 1, the medical instrument 120 is implemented as a scope (such as an endoscope) and the medical instrument 170 is implemented as a needle. Thus, for ease of discussion, the medical instrument 120 is referred to as “the scope” or “the lumen-based medical instrument,” and the medical instrument 170 is referred to as “the needle” or “the percutaneous medical instrument.” However, the medical instrument 120 and the medical instrument 170 can each be implemented as any suitable type of medical instrument including, for example, a scope, a needle, a catheter, a guidewire, a lithotripter, a basket retrieval device, forceps, a vacuum, a needle, a scalpel, an imaging probe, jaws, scissors, graspers, needle holder, micro dissector, staple applier, tacker, suction or irrigation tool, or clip applier, among other examples. In some implementations, a medical instrument may be a steerable device. In some other implementations, a medical instrument may be a non-steerable device. A surgical tool may refer to any device that is configured to puncture or be inserted through the human anatomy, such as a needle, a scalpel, or a guidewire, among other examples. However, a surgical tool can refer to other types of medical instruments.

In some aspects, a medical instrument, such as the scope 120 and/or the needle 170, may include a sensor that is configured to generate sensor data, which can be sent to another device. In some implementations, the sensor data may indicate a pose (including a location and/or orientation) of the medical instrument and/or can be used to determine a pose of the medical instrument. For example, a sensor can include an electromagnetic (EM) sensor with a coil of conductive material. The EM field generator 180 can provide an EM field that is detected by the EM sensor on the medical instrument. The magnetic field can induce small currents in coils of the EM sensor, which can be analyzed to determine a distance and/or angle or orientation between the EM sensor and the EM field generator. In some other implementations, a medical instrument can include other types of sensors configured to generate sensor data, such as a camera, a range sensor, a radar device, a shape sensing fiber, an accelerometer, a gyroscope, an accelerometer, a satellite-based positioning sensor (such as a global positioning system (GPS)), or a radio-frequency transceiver, among other examples. In some implementations, a sensor may be positioned on a distal end of a medical instrument. In some implementations, a sensor on a medical instrument may provide sensor data to the control system 140 and the control system 140 may perform one or more localization techniques to determine or track a position and/or an orientation of the medical instrument.

The terms “scope” and “endoscope” are used herein according to their broad and ordinary meanings and can refer to any type of elongate medical instrument having image generating, viewing, and/or capturing functionality and configured to be introduced into any type of organ, cavity, lumen, chamber, and/or space of a body. For example, references herein to scopes or endoscopes can refer to a ureteroscope (such as for accessing the urinary tract), a laparoscope, a nephroscope (such as for accessing the kidneys), a bronchoscope (such as for accessing an airway, such as the bronchus), a colonoscope (such as for accessing the colon), an arthroscope (such as for accessing a joint), a cystoscope (such as for accessing the bladder), or a borescope, among other examples.

A scope can comprise a tubular and/or flexible medical instrument that is configured to be inserted into the anatomy of a patient to capture images of the anatomy. In some implementations, a scope may accommodate wires and/or optical fibers to transfer signals to or from an optical assembly and a distal end of the scope, which can include an imaging device, such as an optical camera. The camera or imaging device can be used to capture images of an internal anatomical space, such as a calyx or papilla of a kidney. A scope can further accommodate optical fibers to carry light from proximately-located light sources, such as light-emitting diodes, to the distal end of the scope. The distal end of the scope can include ports for light sources to illuminate an anatomical space when using the camera or imaging device. In some implementations, the scope may be controlled by a robotic system, such as the robotic system 110. The imaging device can comprise an optical fiber, fiber array, and/or lens. The optical components can move along with the tip of the scope such that movement of the tip of the scope results in changes to the images captured by the imaging device.

A scope can be articulable, such as with respect to at least a distal portion of the scope, so that the scope can be steered within the human anatomy. In some implementations, a scope may be articulated with, for example, five or six degrees of freedom, including X, Y, Z coordinate movement, as well as pitch, yaw, and roll. A position sensor(s) of the scope can likewise have similar degrees of freedom with respect to the position information they produce or provide. A scope can include telescoping parts, such as an inner leader portion and an outer sheath portion, which can be manipulated to telescopically extend the scope. In some aspects, a scope may comprise a rigid or flexible tube configured to be passed within an outer sheath, catheter, introducer, or other lumen-type device, or can be used without such devices. In some implementations, a scope may include a working channel for deploying medical instruments (such as lithotripters, basketing devices, or forceps), irrigation, and/or aspiration to an operative region at a distal end of the scope.

The robotic system 110 can be configured to at least partly facilitate execution of a medical procedure. The robotic system 110 can be arranged in a variety of ways depending on the particular procedure. The robotic system 110 can include the one or more robotic arms 112 configured to engage with and/or control the scope 120 to perform a procedure. As shown, each robotic arm 112 can include multiple arm segments coupled to joints, which can provide multiple degrees of movement. In the example of FIG. 1, the robotic system 110 is positioned proximate to the patient’s legs and the robotic arms 112 are actuated to engage with and position the scope 120 for access into an access point, such as the urethra of the patient 130. When the robotic system 110 is properly positioned, the scope 120 can be inserted into the patient 130 robotically using the robotic arms 112, manually by the physician 160, or a combination thereof. The robotic arms 112 also can be connected to the EM field generator 180, which can be positioned near a treatment site, such as within proximity to the kidneys of the patient 130.

The robotic system 110 can include a support structure 114 coupled to the one or more robotic arms 112. The support structure 114 can include control electronics or circuitry, one or more power sources, one or more pneumatics, one or more optical sources, one or more actuators (such as motors to move the one or more robotic arms 112), memory or data storage, and/or one or more communication interfaces. In some implementations, the support structure 114 includes an input/output (I/O) device(s) 116 configured to receive input, such as user input to control the robotic system 110, and/or provide output, such as a graphical user interface (GUI), information regarding the robotic system 110, or information regarding a procedure, among other examples. The I/O device(s) 116 can include a display, a touchscreen, a touchpad, a projector, a mouse, a keyboard, a microphone, a speaker, etc. In some implementations, the robotic system 110 is movable (such as the support structure 114 includes wheels) so that the robotic system 110 can be positioned in a location that is appropriate or desired for a procedure. In other implementations, the robotic system 110 is a stationary system. Further, in some implementations, the robotic system 110 is integrated into the table 150.

The robotic system 110 can be coupled to any component of the medical system 100, such as the control system 140, the table 150, the EM field generator 180, the scope 120, and/or the needle 170. In some implementations, the robotic system is communicatively coupled to the control system 140. In one example, the robotic system 110 can be configured to receive a control signal from the control system 140 to perform an operation, such as to position a robotic arm 112 in a particular manner, or manipulate the scope 120, among other examples. In response, the robotic system 110 can control a component of the robotic system 110 to perform the operation. In another example, the robotic system 110 is configured to receive an image from the scope 120 depicting internal anatomy of the patient 130 and/or send the image to the control system 140, which can then be displayed on the display(s) 142. Furthermore, in some implementations, the robotic system 110 is coupled to a component of the medical system 100, such as the control system 140, in such a manner as to allow for fluids, optics, power, or the like to be received therefrom.

The control system 140 can be configured to provide various functionality to assist in performing a medical procedure. In some implementations, the control system 140 can be coupled to the robotic system 110 and operate in cooperation with the robotic system 110 to perform a medical procedure on the patient 130. For example, the control system 140 can communicate with the robotic system 110 via a wireless or wired connection (such as to control the robotic system 110 and/or the scope 120, receive images captured by the scope 120), provide power to the robotic system 110 via one or more electrical connections, or provide optics to the robotic system 110 via one or more optical fibers or other components, among other examples. Further, in some implementations, the control system 140 may communicate with the needle 170 and/or the scope 120 to receive sensor data from the needle 170 and/or the scope 120 (via the robotic system 110 and/or directly from the needle 170 and/or the scope 120). In some implementations, the control system 140 may communicate with the table 150 to position the table 150 in a particular orientation or otherwise control the table 150. Further, in some implementations, the control system 140 may communicate with the EM field generator 180 to control generation of an EM field around the patient 130.

The control system 140 includes various I/O devices configured to assist the physician 160 or others in performing a medical procedure. In this example, the control system 140 includes an I/O device(s) 146 that is employed by the physician 160 or other user to control the scope 120, such as to navigate the scope 120 within the patient 130. For example, the physician 160 can provide input via the I/O device(s) 146 and, in response, the control system 140 can send control signals to the robotic system 110 to manipulate the scope 120. Although the I/O device(s) 146 is illustrated as a controller in the example of FIG. 1, the I/O device(s) 146 can be implemented as a variety of types of I/O devices, such as a touchscreen, a touch pad, a mouse, or a keyboard, among other examples.

As described above, the display(s) 142 can provide a graphical interface 144 to assist the physician 160 in manipulating the needle 170. The display(s) 142 can also provide (such as via the graphical interface 144 and/or another interface) information regarding the scope 120. For example, the control system 140 can receive real-time images that are captured by the scope 120 and display the real-time images via the display(s) 142. Additionally, or alternatively, the control system 140 can receive signals (such as analog, digital, electrical, acoustic or sonic, pneumatic, tactile, or hydraulic signals) from a medical monitor and/or a sensor associated with the patient 130, and the display(s) 142 can present information regarding the health or environment of the patient 130. Such information can include information that is displayed via a medical monitor including, for example, a heart rate (such as ECG or HRV), blood pressure or rate, muscle bio-signals (such as EMG), body temperature, blood oxygen saturation (such as SpO2), CO2, brain waves (such as EEG), or environmental temperatures, among other examples.

To facilitate the functionality of the control system 140, the control system 140 can include various components or subsystems. For example, the control system 140 can include control electronics or circuitry, as well as one or more power sources, pneumatics, optical sources, actuators, memory or data storage devices, and/or communication interfaces. In some implementations, the control system 140 may include control circuitry comprising a computer-based control system that is configured to store executable instructions, that when executed, cause various operations to be implemented. In some implementations, the control system 140 may be movable (such as in FIG. 1). In some other implementations, the control system 140 may be a stationary system. Although various functionality and components are discussed as being implemented by the control system 140, any such functionality and/or components can be integrated into and/or performed by other systems and/or devices, such as the robotic system 110, the table 150, and/or the EM generator 180 (or even the scope 120 and/or the needle 170).

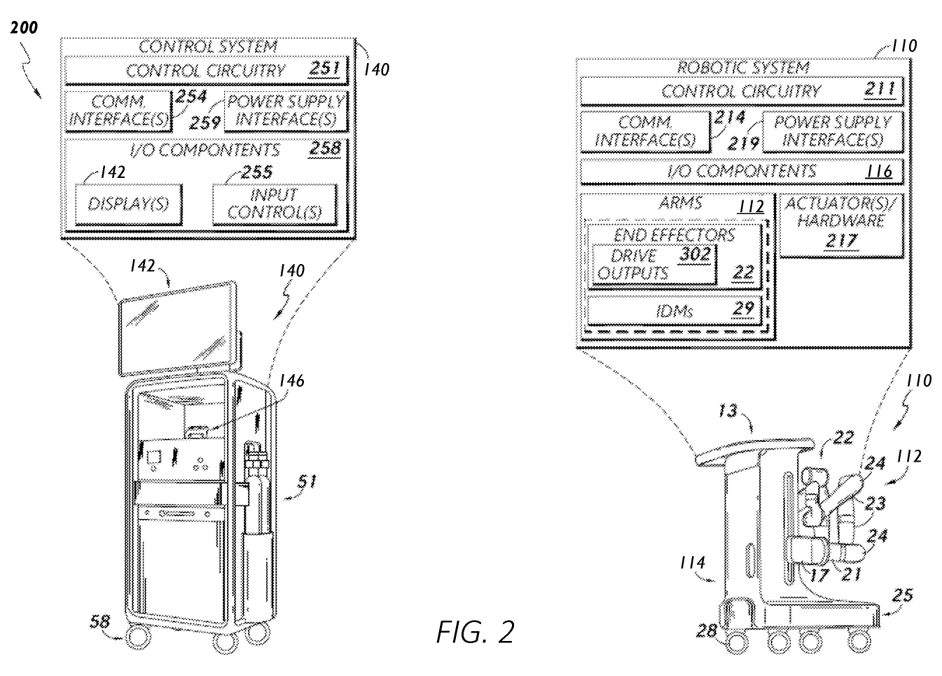

FIG. 2 shows another example medical system 200, according to some implementations. In some implementations, the medical system 200 may be one example of the medical system 100 of FIG. 1. For example, the medical system 200 is shown to include the robotic system 110 and the control system 140 of FIG. 1.

With reference to FIG. 2, the robotic system 110 includes an elongated support structure 114 (also referred to as a “column”), a robotic system base 25, and a console 13 at the top of the column 114. The column 114 may include one or more arm supports 17 (also referred to as a “carriage”) for supporting the deployment of the one or more robotic arms 112. The arm support 17 may include individually-configurable arm mounts that rotate along a perpendicular axis to adjust the base of the robotic arms 112 for better positioning relative to the patient. The robotic arms 112 may be configured to engage with and/or control the scope 120 and/or the needle 170 to perform one or more aspects of a medical procedure. For example, a scope-advancement instrument coupling (such as an instrument device manipulator) can be attached to the distal portion of one of the arms 112, to facilitate robotic control or advancement of the scope 120, while another one of the arms 112 may have associated therewith an instrument coupling that is configured to facilitate advancement of the needle 170.

The arm support 17 also includes a column interface that allows the arm support 17 to vertically translate along the column 114. In some implementations, the column interface can be connected to the column 114 through slots that are positioned on opposite sides of the column 114 to guide the vertical translation of the arm support 17. The slot contains a vertical translation interface to position and hold the arm support 17 at various vertical heights relative to the robotic system base 25. Vertical translation of the arm support 17 allows the robotic system 110 to adjust the reach of the robotic arms 112 to meet a variety of table heights, patient sizes, and physician preferences. Similarly, the individually-configurable arm mounts on the arm support 17 can allow the robotic arm base 21 of the robotic arms 112 to be angled in a variety of configurations.

The robotic arms 112 may generally comprise robotic arm bases 21 and end effectors 22, separated by a series of linkages 23 that are connected by a series of joints 24, each joint 24 comprising one or more independent actuators 217. Each actuator 217 may comprise an independently-controllable motor. Each independently-controllable joint 24 can provide an independent degree of freedom of movement to the robotic arm. In some implementations, each of the arms 112 has seven joints, and thus provides seven degrees of freedom, including “redundant” degrees of freedom. Redundant degrees of freedom allow the robotic arms 112 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The robotic system base 25 balances the weight of the column 114, arm support 17, and arms 112 over the floor. Accordingly, the robotic system base 25 may house certain relatively heavier components, such as electronics, motors, power supply, as well as components that selectively enable movement or immobilize the robotic system. For example, the robotic system base 25 can include wheel-shaped casters 28 that allow for the robotic system to easily move around the operating room prior to a procedure. After reaching the appropriate position, the casters 28 may be immobilized using wheel locks to hold the robotic system 110 in place during the procedure.

A console 13 is positioned at the upper end of column 114 and can provide one or more I/O components 116, such as a user interface for receiving user input and a display screen (or a dual-purpose device such as, for example, a touchscreen) to provide the physician or user with preoperative and intra-operative data. Example pre-operative data may include pre-operative plans, navigation and mapping data derived from pre-operative computed tomography (CT) scans, and/or notes from pre-operative patient interviews. Example intra-operative data may include optical information provided from the tool, sensor and coordinate information from sensors, as well as vital patient statistics, such as respiration, heart rate, and/or pulse. The console 13 may be positioned and tilted to allow a physician to view the console 13, robotic arms 112, and patient while operating the console 13 from behind the robotic system 110.

The end effector 22 of each of the robotic arms 112 may comprise an instrument device manipulator (IDM) 29, which may be attached using a mechanism changer interface (MCI). In some implementations, the IDM 29 can be removed and replaced with a different type of IDM, for example, a first type of IDM may manipulate a scope, while a second type of IDM may manipulate a needle. Another type of IDM may be configured to hold an electromagnetic field generator (such as the EM field generator 180). An MCI can include connectors to transfer pneumatic pressure, electrical power, electrical signals, and/or optical signals from the robotic arm 112 to the IDM 29. The IDMs 29 may be configured to manipulate medical instruments, such as the scope 120, using techniques including, for example, direct drives, harmonic drives, geared drives, belts and pulleys, magnetic drives, and the like. In some implementations, the IDMs 29 can be attached to respective ones of the robotic arms 112, wherein the robotic arms 112 are configured to insert or retract the respective coupled medical instruments into or out of the treatment site. The robotic system 110 further includes power 219 and communication 214 interfaces (such as connectors) to transfer pneumatic pressure, electrical power, electrical signals, and/or optical signals from the robotic arms 112 to the IDMs 29.

In some implementations, a user can manually manipulate a robotic arm 112 of the robotic system 110 without using electronic user controls. For example, during setup in a surgical operating room, a user may move the robotic arms 112 and/or any other medical instruments to provide desired access to a patient. The robotic system 110 may rely on force feedback and inertia control from the user to determine appropriate configuration of the robotic arms 112 and associated instrumentation.

As described with reference to FIG. 1, the medical system 100 can include control circuitry configured to perform certain functionality described herein, including control circuitry 211 of the robotic system 110 and/or control circuitry 251 of the control system 140. That is, the control circuitry of the medical system 100 may be part of the robotic system 110, the control system 140, or some combination thereof. Therefore, any reference herein to control circuitry may refer to circuitry embodied in a robotic system, a control system, or any other component of a medical system, such as the medical system 100 shown in FIG. 1. The term “control circuitry” is used herein according to its broad and ordinary meaning, and may refer to any collection of processors, processing circuitry, processing modules or units, chips, dies (such as semiconductor dies including come or more active and/or passive devices and/or connectivity circuitry), microprocessors, micro-controllers, digital signal processors, microcomputers, central processing units, field programmable gate arrays, programmable logic devices, state machines (such as hardware state machines), logic circuitry, analog circuitry, digital circuitry, and/or any device that manipulates signals (analog and/or digital) based on hard coding of the circuitry and/or operational instructions.

Control circuitry referenced herein may further include one or more circuit substrates (such as printed circuit boards), conductive traces and vias, and/or mounting pads, connectors, and/or components. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. In implementations where control circuitry comprises a hardware and/or software state machine, analog circuitry, digital circuitry, and/or logic circuitry, data storage device(s) or register(s) storing any associated operational instructions may be embedded within, or external to, the circuitry comprising the state machine, analog circuitry, digital circuitry, and/or logic circuitry.

The control circuitry 211 and/or 251 may comprise a computer-readable medium storing, and/or configured to store, hard-coded and/or operational instructions corresponding to at least some of the steps and/or functions illustrated in one or more of the present figures and/or implementations described herein. Such computer-readable medium can be included in an article of manufacture in some instances. The control circuitry 211 and/or 251 may be locally maintained on the robotic system 110 or the control system 140 or may be remotely located at least in part (such as communicatively coupled indirectly via a local area network and/or a wide area network). Any of the control circuitry 211 and/or 251 may be configured to perform any aspect(s) of the various processes disclosed herein.

With respect to the robotic system 110, at least a portion of the control circuitry 211 may be integrated with the base 25, column 114, and/or console 13 of the robotic system 110, and/or another system communicatively coupled to the robotic system 110. With respect to the control system 140, at least a portion of the control circuitry 251 may be integrated with a console base 51 and/or display 142 of the control system 140. It should be understood that any description herein of functional control circuitry or associated functionality may be embodied in the robotic system 110, the control system 140, or any combination thereof, and/or at least in part in one or more other local or remote systems or devices.

With further reference to FIG. 2, the control system 140 can include various I/O components 258 configured to assist the physician or others in performing a medical procedure. For example, the I/O components 258 can be configured to allow for user input to control or navigate the scope 120 and/or needle 170 within the patient. In some implementations, the physician can provide input to the control system 140 and/or robotic system 110 via one or more input controls 255, wherein in response to such input, control signals can be sent to the robotic system 110 to manipulate the scope 120 and/or needle 170. Example suitable input controls 255 may include any type of user input devices or device interfaces, such as buttons, keys, joysticks, handheld controllers (such as video-game type controllers), computer mice, trackpads, trackballs, control pads, foot pedals, sensors (such as motion sensors or cameras) that capture hand or finger gestures, or touchscreens, among other examples. To facilitate the functionality of the control system 140, the control system can include various components (sometimes referred to as “subsystems”). For example, the control system 140 can include control electronics or circuitry 251, as well as one or more power supplies or supply interfaces 259, pneumatic devices, optical sources, actuators, data storage devices, and/or communication interfaces 254.

The various components of the medical system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. For example, the communication interfaces 214 and 254 of the robotic system 110 and the control system 140, respectively, can be configured to communicate with one or more devices, sensors, or systems, such as over a wireless and/or wired network connection. In some implementations, the various communication interfaces can implement a wireless technology such as Bluetooth, Wi-Fi, near field communication (NFC), or the like. Furthermore, in some implementations, the various components of the system 100 can be connected for data communication, fluid exchange, power exchange, and so on via one or more support cables, tubes, or the like.

With further reference to FIG. 1, the medical system 100 can provide a variety of benefits, such as providing guidance to assist a physician in performing a procedure (such as instrument tracking or instrument alignment information), enabling a physician to perform a procedure from an ergonomic position without the need for awkward arm motions and/or positions, enabling a single physician to perform a procedure with one or more medical instruments, avoiding radiation exposure (such as associated with fluoroscopy techniques), enabling a procedure to be performed in a single-operative setting, or providing continuous suction to remove an object more efficiently (such as to remove a kidney stone), among other examples. For example, the medical system 100 can provide guidance information to assist a physician in using various medical instruments to access a target anatomical feature while minimizing bleeding and/or damage to anatomy (such as critical organs or blood vessels).

Further, the medical system 100 can provide non-radiation based navigational and/or localization techniques and/or reduce the amount of equipment in the operating room. Moreover, the medical system 100 can provide functionality that is distributed between at least the control system 140 and the robotic system 110, which can be independently movable. Such distribution of functionality and/or mobility can enable the control system 140 and/or the robotic system 110 to be placed at locations that are optimal for a particular medical procedure, which can maximize working area around the patient and/or provide an optimized location for a physician to perform a procedure.

Although various techniques and systems are discussed as being implemented as robotically-assisted procedures (such as procedures that at least partly use the medical system 100), the techniques and systems can be implemented in other procedures, such as in fully-robotic medical procedures or human-only procedures (such as free of robotic systems). For example, the medical system 100 can be used to perform a procedure without a physician holding or manipulating a medical instrument (such as a fully-robotic procedure). That is, medical instruments that are used during a procedure, such as the scope 120 and the needle 170, can each be held or controlled by components of the medical system 100, such as the robotic arm(s) 112 of the robotic system 110.

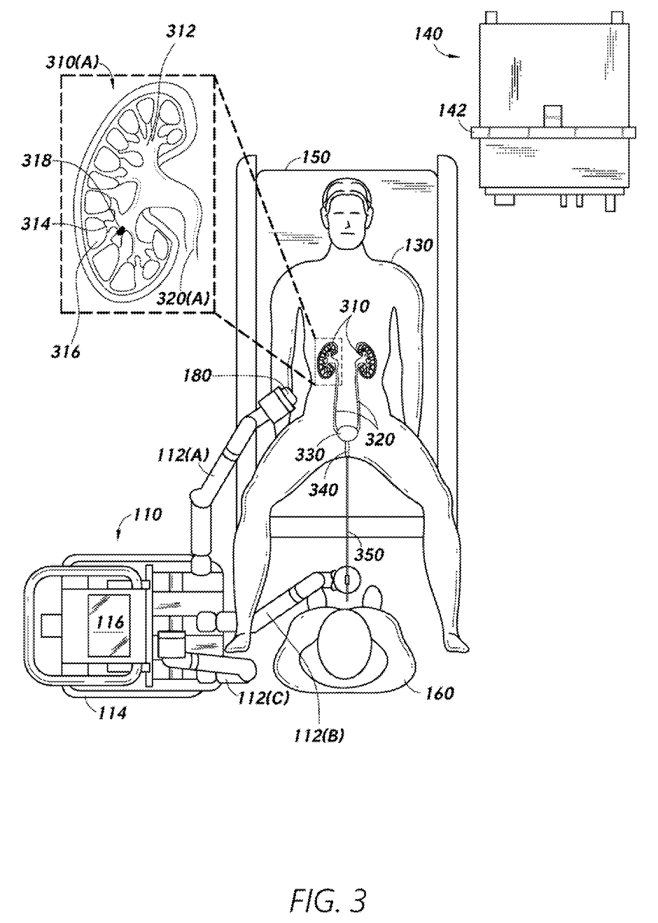

FIGS. 3–5 show an example percutaneous access procedure that can be performed using the medical system 100 of FIG. 1. In these examples, the medical system 100 is arranged in an operating room to remove kidney stones from the patient 130 with the assistance of the scope 120 and the needle 170. In some implementations, the patient 130 may be positioned in a modified supine position with the patient 130 slightly tilted to the side to access the back or side of the patient 130, such as that illustrated in FIG. 1. However, the patient 130 can be positioned in other manners, such as a supine position or a prone position, among other examples. For ease of illustration in viewing the anatomy of the patient 130, FIGS. 3–5 illustrate the patient 130 in a supine position with the legs spread apart. Also, for ease of illustration, the imaging device 190 (including the C-arm) shown in FIG. 1 has been removed.

Although FIGS. 3–5 illustrate use of the medical system 100 to perform a percutaneous access procedure to remove a kidney stone from the patient 130, the medical system 100 can be used to remove a kidney stone in other manners and/or to perform other procedures. Further, the patient 130 can be arranged in other positions as desired for a procedure. Various acts or workflow are described with reference to FIGS. 3–5, and throughout this disclosure, as being performed by the physician 160. It should be understood that these acts can be performed directly by the physician 160, a user under direction of the physician 160, another user (such as a technician), a combination thereof, and/or any other user.

The renal anatomy, as illustrated at least in part in FIGS. 3–5, is described here for reference with respect to certain medical procedures relating to aspects of the present disclosure. The kidneys generally comprise two bean-shaped organs located on the left and right in the retroperitoneal space. The kidneys receive blood from the paired renal arteries, and blood exits into the paired renal veins. Each kidney is attached to a ureter, which is a tube that carries excreted urine from the kidney to the bladder. The bladder is attached to the urethra. A recessed area on the concave border of the kidney is the renal hilum, where the renal artery enters the kidney and the renal vein and ureter leave. The kidney is surrounded by tough fibrous tissue, the renal capsule, which is itself surrounded by perirenal fat, renal fascia, and pararenal fat. The anterior (front) surface of these tissues is the peritoneum, while the posterior (rear) surface is the transversalis fascia.

The functional substance, or parenchyma, of the kidney is divided into two major structures: the outer renal cortex and the inner renal medulla. These structures take the shape of a plurality of cone-shaped renal lobes, each containing renal cortex surrounding a portion of medulla called a renal pyramid. The tip, or papilla, of each pyramid empties urine into a respective minor calyx; minor calyces empty into major calyces, and major calyces empty into the renal pelvis, which transitions to the ureter. At the hilum, the ureter and renal vein exit the kidney and the renal artery enters. Hilar fat and lymphatic tissue with lymph nodes surrounds these structures. The hilar fat is contiguous with a fat-filled cavity called the renal sinus. The renal sinus collectively contains the renal pelvis and calyces and separates these structures from the renal medullary tissue.

FIGS. 3–5 show various features of the anatomy of the patient 130. For example, the patient 130 includes kidneys 310 fluidly connected to a bladder 330 via ureters 320, and a urethra 340 fluidly connected to the bladder 330. As shown in the enlarged depiction of the kidney 310(A), the kidney 310(A) includes calyces (such as a calyx 312), renal papillae (such as a papilla 314), and renal pyramids (such as a pyramid 316). In these examples, a kidney stone 318 is located in proximity to the papilla 314. However, the kidney stone 318 can be located at other locations within the kidney 310(A) or elsewhere.

As shown in FIG. 3, to remove the kidney stone 318 in the example percutaneous procedure, the physician 160 can position the robotic system 110 at the side or foot of the table 150 to initiate delivery of the scope 120 (not shown in FIG. 3) into the patient 130. In particular, the robotic system 110 can be positioned at the side of the table 150 within proximity to the feet of the patient 130 and aligned for direct linear access to the urethra 340 of the patient 130. The hip of the patient 130 may be used as a reference point to position the robotic system 110. Once positioned, one or more of the robotic arms 112, such as the robotic arms 112(B) and 112(C), can stretch outwards to reach in between the legs of the patient 130. As shown in FIG. 3, the robotic arm 112(B) can be controlled to extend and provide linear access to the urethra 340.

In this example, the physician 160 inserts a medical instrument 350 at least partially into the urethra 340 along this direct linear access path (also referred to as a “virtual rail”). The medical instrument 350 can include a lumen-type device configured to receive the scope 130, thereby assisting in inserting the scope 120 into the anatomy of the patient 130. By aligning the robotic arm 112(B) to the urethra 340 of the patient 130 and/or using the medical instrument 350, friction and/or forces on the sensitive anatomy in the area can be reduced. In some other implementations, the scope 120 may be inserted directly into the urethra 340 without the use of the medical instrument 350.

The physician 160 can also position the robotic arm 112(A) near a treatment site for the procedure. For example, the robotic arm 112(A) can be positioned within proximity to the incision site and/or the kidneys 310 of the patient 130. The robotic arm 112(A) can be connected to the EM field generator 180 to assist in tracking a location of the scope 120 and/or the needle 170 during the procedure. Although the robotic arm 112(A) is positioned relatively close to the patient 130, in some embodiments the robotic arm 112(A) is positioned elsewhere and/or the EM field generator 180 is integrated into the table 150 (which can allow the robotic arm 112(A) to be in a docked position). At this point in the procedure, the robotic arm l 12(C) remains in a docked position, as shown in FIG. 3. However, the robotic arm l 12(C) can be used in some implementations to perform any of the functions discussed above of the robotic arms 112(A) and/or 112(C).

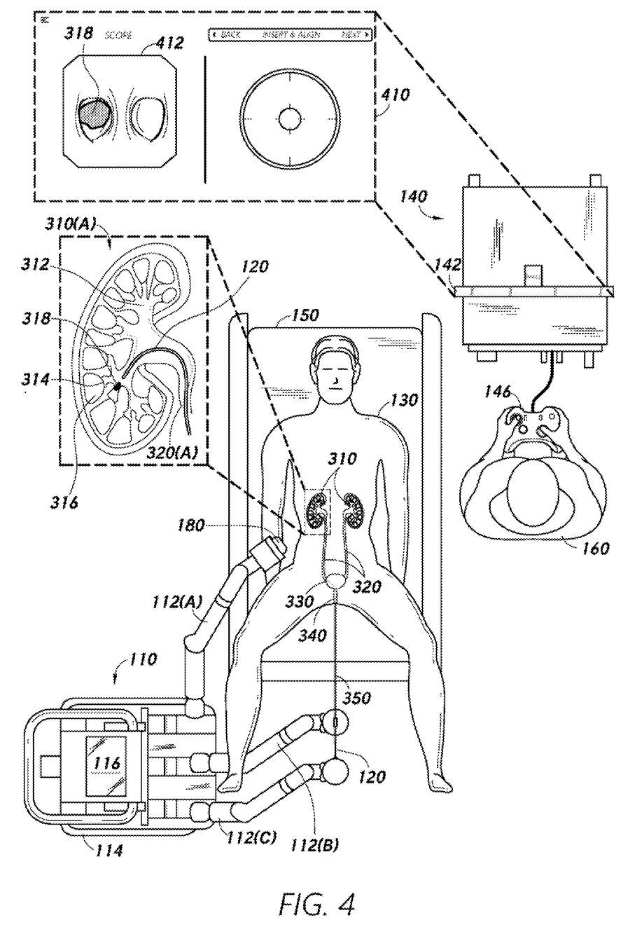

Once the robotic system 110 is properly positioned and/or the medical instrument 350 is inserted at least partially into the urethra 340, the scope 120 can be inserted into the patient 130 robotically, manually, or a combination thereof, as shown in FIG. 4. For example, the physician 160 can connect the scope 120 to the robotic arm 112(C) and/or position the scope 120 at least partially within the medical instrument 350 and/or the patient 130. The scope 120 can be connected to the robotic arm 112(C) at any time, such as before the procedure or during the procedure (such as after positioning the robotic system 110). The physician 160 can then interact with the control system 140, such as the I/O device(s) 146, to navigate the scope 120 within the patient 130. For example, the physician 160 can provide input via the I/O device(s) 146 to control the robotic arm l 12(C) to navigate the scope 120 through the urethra 340, the bladder 330, the ureter 320(A), and up to the kidney 310(A).

In some aspects, the control system 140 may present an instrument-alignment interface 410 (such as the graphical interface 144 of FIG. 1) on the display(s) 142 to view a real-time image 412 captured by the scope 120 to assist the physician 160 in controlling the scope 120. The physician 160 can navigate the scope 120 to locate the kidney stone 318, as depicted in the image 412. In some implementations, the control system 140 may use localization techniques to determine a position and/or an orientation of the scope 120, which can be viewed by the physician 160 via the display(s) 142 to also assist in controlling the scope 120. Further, in some implementations, other types of information can be presented on the display(s) 142 to assist the physician 160 in controlling the scope 120, such as x-ray images of the internal anatomy of the patient 130.

Upon locating the kidney stone 318, the physician 160 can designate a target location for the needle 170 to enter the kidney 310(A) for eventual extraction of the kidney stone 318. For example, to minimize bleeding and/or avoid hitting a blood vessel or other undesirable anatomy of the kidney 310(A) and/or anatomy surrounding the kidney 310(A), the physician 160 can seek to align the needle 170 with an axis of a calyx. To do so, the physician 160 can designate a target location that is aligned with the center of the calyx and the center of a papilla (such as the papilla 314). In some implementations, the physician may designate the target by touching the scope 120 to the papilla 314 (also referred to as a “tag” position) and retracting the scope 120 to a “park” position some distance away from the papilla 314 (such as where the entire papilla 314 is within an FOV of a camera disposed on the scope 120). The control system 140 uses localization techniques to determine the “tag” and “park” positions of the scope 120 (such as based on sensor data from an EM sensor disposed on the scope 120) and sets the target location (also referred to as the “EM target”) midway between the “tag” and “park” positions.

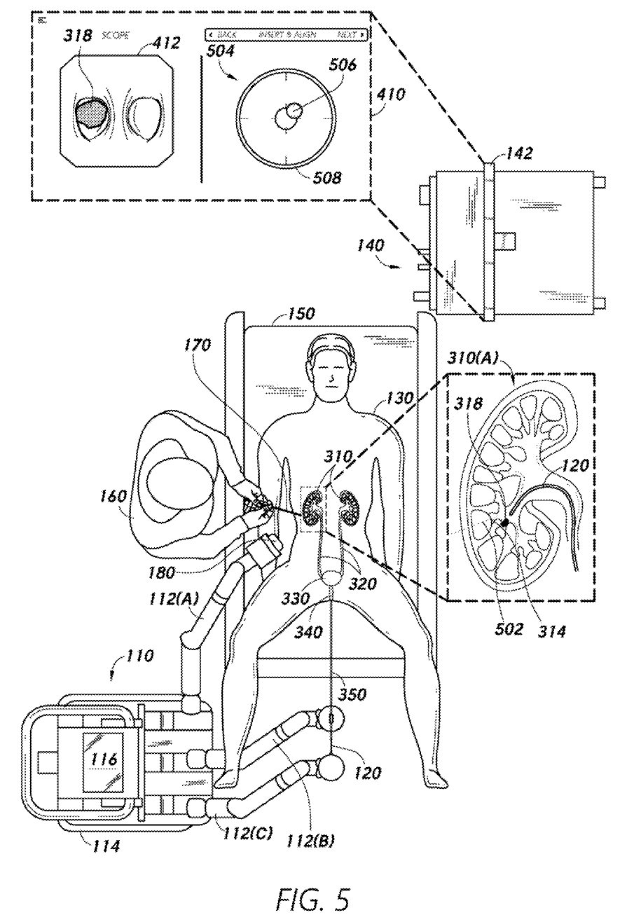

As shown in FIG. 5, the physician 160 can proceed with the procedure by positioning the needle 170 for insertion into the target location. In some implementations, the physician 160 may use his or her best judgment to place the needle 170 on the patient 130 at an incision site, such as based on knowledge regarding the anatomy of the patient 130, experience from previously performing the procedure, an analysis of CT or x-ray images, or other pre-operative information of the patient 130, among other examples. The physician 160 can attempt to avoid critical anatomy of the patient 130, such as the lungs, pleura, colon, paraspinal muscles, ribs, and/or intercostal nerves. In some implementations, the control system 140 may use CT, x-ray, or ultrasound images to provide information to the physician 160 regarding a location to place the needle 170 on the patient 130.

The control system 140 can determine a target trajectory 502 for inserting the needle 170 to assist the physician 160 in reaching the target location (such as the papilla 314). The target trajectory 502 can represent a desired path for accessing to the target location. The target trajectory 502 can be determined based on a position of a medical instrument (such as the needle 170 or the scope 120), a target location within the human anatomy, a position and/or orientation of a patient, or the anatomy of the patient (such as the location of organs within the patient relative to the target location), among other examples. In the example of FIG. 5, the target trajectory 502 includes a straight line that passes through the papilla 314 and the needle 170 (extending from a tip of the needle 170 through the papilla 314, such as a point on an axis of the papilla 314).

However, the target trajectory 502 can take other forms, such as a curved line, and/or can be defined in other manners. In some implementations, the needle 170 may be a flexible bevel-tip needle that is configured to curve as the needle 170 is inserted in a straight manner. Such needle can be used to steer around particular anatomy, such as the ribs or other anatomy. Here, the control system 140 can provide information to guide a user, such as to compensate for deviation in the needle trajectory or to maintain the user on the target trajectory. Although the example of FIG. 5 illustrates the target trajectory 502 extending coaxially through the papilla 314, the target trajectory 502 can have another position, angle, and/or form. For example, a target trajectory can be implemented with a lower pole access point, such as through a papilla located below the kidney stone 318 shown in FIG. 5, with a non-coaxial angle through the papilla, which can be used to avoid the hip.

The control system 140 can use the target trajectory 502 to provide an alignment-progress visualization 504 via the instrument-alignment interface 410. For example, the alignment-progress visualization 504 can include an instrument alignment element 506 indicative of an orientation of the needle 170 relative to the target trajectory 502. The physician 160 can view the alignment-progress visualization 504 and orient the needle 170 to the target trajectory 502. When aligned, the physician 160 can insert the needle 170 into the patient 130 to reach the target location. The alignment-progress visualization 504 may include a progress visualization 508 (also referred to as a “progress bar”) indicating a proximity of the needle 170 to the target location. Thus, the instrument-alignment interface 410 can assist the physician 160 in aligning and/or inserting the needle 170 to reach the target location.

Once the needle 170 has reached the target location, the physician 160 can insert another medical instrument (such as a power catheter, vacuum, or nephroscope) into the path created by the needle 170 and/or over the needle 170. The physician 160 can use the other medical instrument and/or the scope 120 to fragment and remove pieces of the kidney stone 318 from the kidney 310(A).

In some implementations, a position of a medical instrument can be represented with a point or point set, and an orientation of the medical instrument can be represented as an angle or offset relative to an axis or plane. For example, a position of a medical instrument can be represented with a coordinate(s) of a point or point set within a coordinate system (such as one or more X, Y, Z coordinates) and/or an orientation of the medical instrument can be represented with an angle relative to an axis or plane for the coordinate system (such as angle with respect to the X-axis or plane, Y-axis or plane, and/or Z axis or plane). Here, a change in orientation of the medical instrument can correspond to a change in an angle of the medical instrument relative to the axis or plane. Further, in some implementations, an orientation of a medical instrument is represented with yaw, pitch, and/or roll information.

In some implementations, a trajectory may represent a pose. For example, a trajectory of a medical instrument can refer to a pose of the medical instrument, including or indicating both a position and orientation of the medical instrument. Similarly, a target trajectory can refer to a target pose, including or indicating both a position and orientation of a desired path. In some other implementations, a trajectory may refer to either an orientation or a position.

Although particular robotic arms of the robotic system 110 are illustrated as performing particular functions in the context of FIGS. 3-5, any of the robotic arms 112 can be used to perform the functions. Further, any additional robotic arms and/or systems can be used to perform the procedure. Moreover, the robotic system 110 can be used to perform other parts of the procedure. For example, the robotic system 110 can be controlled to align and/or insert the needle into the patient 130. To illustrate, one of the robotic arms 112 can engage with and/or control the needle 170 to position the needle 170 at the appropriate location, align the needle 170 with the target trajectory, and/or insert the needle 170 to the target location. The control system 140 can use localization techniques to perform such processing. Thus, in some implementations, a percutaneous procedure can be performed entirely or partially with the medical system 100 (such as with or without the assistance of the physician 160).

As described with reference to FIGS. 3–5, a percutaneous access procedure can be subdivided into 3 phases: a target selection phase (where a target location within an anatomy is selected or designated for percutaneous access), a site selection phase (where a needle is placed on the surface of the patient’s skin and aligned with the target location), and a needle insertion phase (where the needle is driven percutaneously to rendezvous with the target location). In some implementations of the target selection phase, an EM sensor disposed on the distal end of a scope is used to designate the target location for percutaneous access (such as by touching the scope to a portion of an anatomy and retracting the scope to a park position). EM sensors offer greater positional accuracy compared to other sensing technologies (such as shape sensing) that can also be used for localization. However, adding EM sensors to an endoscope can significantly increase the manufacturing cost and/or complexity of the scope. As a result, many existing endoscopes do not have integrated EM sensors and thus may not be suitable for performing the target localization procedure described with reference to FIG. 4.

On the other hand, many existing endoscopes have working channels in which various other tools can be inserted (such as lithotripters, basket retrieval devices, or forceps). Aspects of the present disclosure recognize that the working channel of an endoscope can be used to introduce one or more sensors into an anatomy for designating a target location. The sensors may implement any sensing technology (such as EM) suitable for tracking the positions of the sensors in a given coordinate space. In some aspects, the position sensors may be coupled to or disposed on an elongate member (such as a flexible wire or shaft) to form a “working channel instrument” that can be inserted through the working channel of an endoscope. The elongate member can be used to manipulate or otherwise position the sensors at or around the target location so that the target location can be detected and recorded for percutaneous access. In this way, the working channel instrument can provide a more direct and/or accurate means of designating a target location within an anatomy.

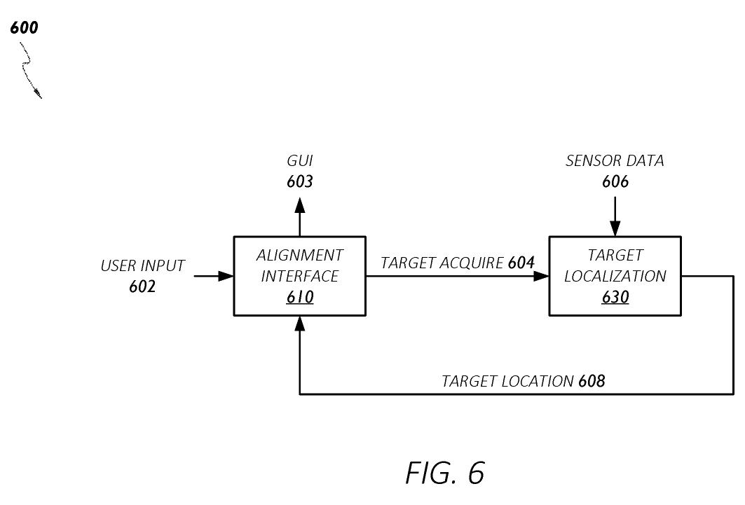

FIG. 6 shows a block diagram of an example target localization system 600, according to some implementations. In some implementations, the target localization system 600 may be one example of any of the control circuitry 251 or 211 of FIG. 2. The target localization system 600 is configured to determine or designate a target location 608 within an anatomy based, at least in part, on sensor data 605 received from one or more position sensors disposed on a working channel instrument. The working channel instrument is any instrument that can be inserted into the anatomy via a working channel of an endoscope and tracked via the sensor data 605. For example, the positions of the sensors disposed on the working channel instrument can be detected and/or monitored using one or more sensing modalities (such as EM).

The target localization system 600 includes an alignment interface component 610 and a target localization component 620. The alignment interface component 610 is configured to display or generate a graphical user interface (GUI) 603 that provides guidance for navigating the endoscope within the anatomy. In some implementations, the GUI 603 may include real-time images or video of the anatomy based on images captured by a camera inside the anatomy. For example, the camera may be disposed on the distal end of the endoscope or working channel instrument. More specifically, the images may depict a portion of the anatomy in an FOV of the camera based on the current instrument pose. In some implementations, the GUI 603 may be one example of the graphical interface 144 of FIG. 1. With reference for example to FIG. 1, the GUI 603 may be presented on the display 142. The alignment interface component 610 is also configured to receive user input 602 via one or more input devices (such as the input controls 255 of FIG. 2). Example suitable input devices may include touchscreens, touchpads, buttons, switches, mice, keyboards, keypads, joysticks, and scroll wheels, among other examples.

In some implementations, the user inputs 602 may include instructions or commands for driving or manipulating the instrument within the anatomy. For example, a user may control the endoscope via the user inputs 602 to reach a target location for percutaneous access (such as a calyx or a papilla). More specifically, the user may navigate the endoscope using the GUI 603 and park the endoscope proximate to the target location. For example, the user may park the endoscope so that that the target location is within the FOV of the camera or otherwise reachable by the working channel instrument. In some aspects, the user may insert the working channel instrument through the working channel of the endoscope once the scope is parked at the desired location. In some implementations, the working channel instrument may be manually inserted through the working channel of the scope (such as under direct control of the user). In some other implementations, the working channel instrument may be coupled to one or more robotic actuators configured to control insertion of the working channel instrument through the working channel of the scope.

The working channel instrument is configured to be inserted so that the position sensors are in contact with, or proximate to, the target location. In some implementations, the working channel instrument may include a position sensor disposed on a flexible wire that can be used to manipulate the sensor within the anatomy. For example, the sensor may be disposed on the distal tip of the wire so that the wire can be used to press and/or hold the sensor in contact with an anatomical surface (such as a papilla). In some other implementations, the working channel instrument may include multiple position sensors disposed along a length of flexible wire that can coil or otherwise deform around an anatomical volume (such as a calyx). Still further, in some implementations, the working channel instrument may include a position sensor disposed on a shaft having a barbed tip that can penetrate and/or latch on to an anatomical surface (such as a papilla). In some aspects, the user may track the movement and/or position of the working channel instrument via the GUI 603 (such as based on the images captured by the camera inside the anatomy), for example, to ensure that the instrument is properly positioned relative to the target location. In some implementations, the GUI 603 may display an anatomical map that indicates the positions of the sensors disposed on the working channel instrument relative to the anatomy.

When the working channel instrument is inserted to the desired position within the anatomy, the user may provide additional user input 602 signaling that a target is ready to be acquired 604. The target localization component 620 responds to the target acquire signal 604 by capturing or otherwise acquiring sensor data 606 indicating the current positions of the sensors disposed on the working channel instrument and determines the target location 608 based on the sensor data 606. In some implementations, where the working channel instrument includes a single sensor disposed on a flexible wire, the position of the sensor when the target acquire signal 604 is detected may be used to designate the target location 608. In some other implementations, where the working channel instrument includes multiple sensors disposed along a length of flexible wire, the centroid of the sensors surrounding the anatomical volume may be used to designate the target location 608. Still further, in implementations, where the working channel instrument includes a shaft having a barbed tip, the position of the sensor while the barb is latched to the anatomical surface may be used to designate the target location 608.

The alignment interface component 610 can further provide guidance (to a user or a robotic system) for inserting a percutaneous access needle into the anatomy based on the target location 608 (such as described with reference to FIG. 5). For example, the alignment interface component 610 may display, on the GUI 603, a spatial relationship between the needle and the target location 608 (such as the instrument-alignment interface 410 of FIGS. 4 and 5). The alignment interface component 610 can determine the pose of the needle based on sensor data received from one or more position sensors disposed on the needle (not shown for simplicity). In some implementations, the position sensors of the needle may implement the same sensing technology as the position sensors of the working channel instrument. In such implementations, the poses of the needle and the working channel instrument can be tracked in the same sensor space (such as the EM space). In some other implementations, the position sensors of the needle may implement a different sensing technology than the position sensors of the working channel instrument. In such implementations, the alignment interface component 610 may register the sensor space of the needle with the sensor space of the working channel instrument to display the poses of the needle and the working channel instrument in a common coordinate frame.

By leveraging the sensor data 606 from a working channel instrument to designate the target location 608, the target localization system 600 can significantly reduce the cost of tools needed to perform a percutaneous access procedure. For example, the working channel instruments of the present disclosure are generally much less complex and thus cheaper to manufacture than articulable endoscopes with integrated sensors. Such working channel instruments can be used in conjunction with many existing endoscopes that have working channels or lumens and existing means for detecting the position sensors (such as an EM field generator), thereby reducing the need for specialized equipment to perform a percutaneous access procedure. Further, because the working channel instrument can be placed in contact with the anatomy, any changes or movements of the anatomy (such as due to respiration) may be reflected in the sensor data 606. As a result, the target location 608 tracks the movements of the anatomy, which can significantly improve the outcome of a percutaneous access procedure. For example, the target location 608 can provide greater coaxial alignment with the surrounding anatomy (such as a calyx) which can improve procedure efficiency, resulting in less trauma to the tissue, less time under anesthesia, and/or fewer access attempts.

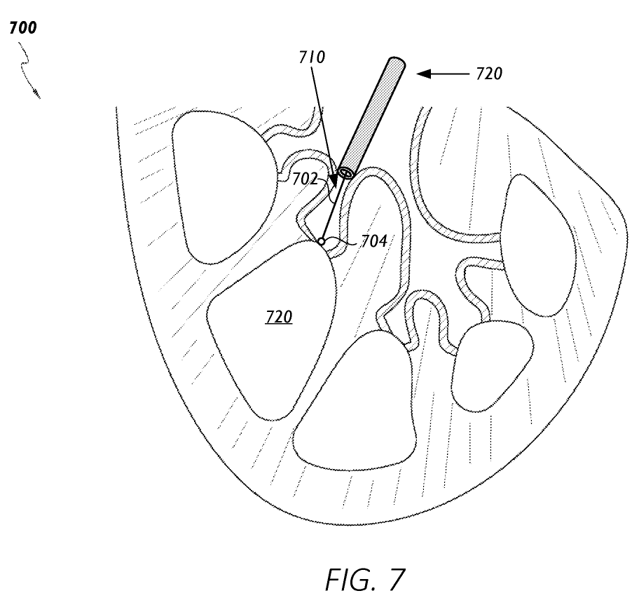

FIG. 7 shows an example positioning of a working channel instrument 710 for designating a target for percutaneous access in an anatomy 700, according to some implementations. In the example of FIG. 7, the anatomy 700 is depicted as a kidney. However, the working channel instrument 710 can be used to designate a target location in other types of anatomies using the same or similar techniques as described with reference to FIG. 7.

In the example of FIG. 7, the working channel instrument 710 includes a sensor 704 disposed on a flexible wire 702. The sensor 704 can be coupled or attached to the flexible wire 702 using any suitable means (including adhesives, laser welding, or other mechanical means). The flexible wire 702 can have any shape or geometry suitable for insertion through the working channel of an endoscope 720. More specifically, the flexible wire 702 can be formed from any flexible or deformable material that can bend or deflect when contacting surfaces of the anatomy 700, for example, to avoid puncturing or damaging the anatomy 700.

As shown in FIG. 7, the endoscope 720 is positioned proximate to a target anatomical feature 720 (such as a papilla) through which a percutaneous access needle is to be inserted. In some implementations, the endoscope 720 may be one example of the scope 120 of FIGS. 1–5. The flexible wire 702 extends beyond the distal opening of the endoscope 720 so that the sensor 704 is exposed outside the working channel of the endoscope 720. In the example of FIG. 7, the sensor 704 is shown coupled or attached to the distal tip of the wire 702 and in contact with the surface of the papilla 720. In some implementations, the flexible wire 702 may bend or deflect when the sensor 704 is pressed against the papilla 720 (such as to ensure that the sensor 704 is held firmly against the surface of the papilla 720). In some other implementations, the sensor 704 may be offset from the distal tip of the wire 702 (such as by a predetermined distance), and thus, not in contact with surface of the papilla 720.

The sensor 704 may implement any suitable sensing technology that can be used to determine its position in a coordinate system. In some implementations, the sensor 704 may be an EM sensor that can be tracked in relation to an EM field (such as produced by the EM field generator 180 of FIGS. 1 and 3–5). For example, the sensor 704 may produce sensor data indicating its position in a given coordinate frame (such as the EM sensor space). In some implementations, the sensor data produced by the sensor 704 may be one example of the sensor data 606 of FIG. 6. More specifically, with reference to FIG. 6, the position of the sensor 704 while in contact with the papilla 720 may be designated the target location 608.

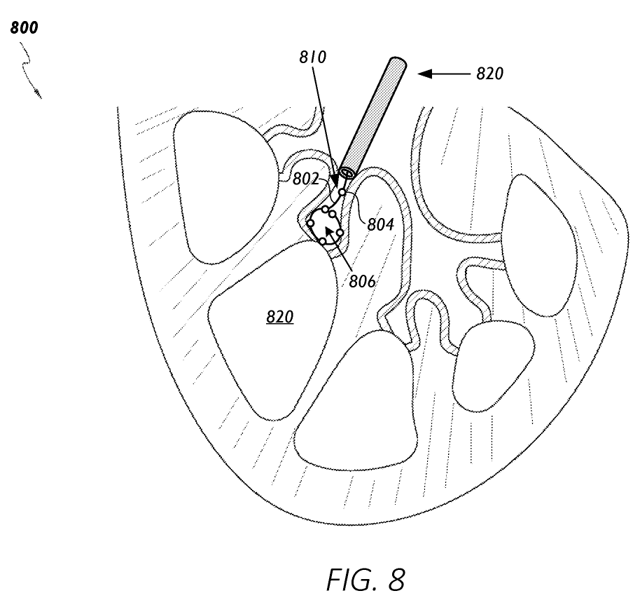

FIG. 8 shows another example positioning of a working channel instrument 810 for designating a target for percutaneous access in an anatomy 800, according to some implementations. In the example of FIG. 8, the anatomy 800 is depicted as a kidney. However, the working channel instrument 810 can be used to designate a target location in other types of anatomies using the same or similar techniques as described with reference to FIG. 8.

In the example of FIG. 8, the working channel instrument 810 includes multiple sensors 804 disposed along the length of a flexible wire 802. The sensors 804 can be coupled or attached to the flexible wire 802 using any suitable means (including adhesives, laser welding, or other mechanical means). The flexible wire 802 can have any shape or geometry suitable for insertion through the working channel of an endoscope 820. More specifically, the flexible wire 802 can be formed from any flexible or deformable material that can bend or deflect when contacting surfaces of the anatomy 800, for example, to avoid puncturing or damaging the anatomy 800.

As shown in FIG. 8, the endoscope 820 is positioned proximate to a target anatomical feature 820 (such as a papilla) through which a percutaneous access needle is to be inserted. In some implementations, the endoscope 820 may be one example of the scope 120 of FIGS. 1–5. The flexible wire 802 extends beyond the distal opening of the endoscope 820 and coils around an anatomical volume (such as a calyx) adjacent to the papilla 820. More specifically, as shown in FIG. 8, the flexible wire 802 can conform to the shape of the calyx so that the sensors 804 are positioned around and/or otherwise fill a space within the calyx. In some implementations, the sensors 804 may be spaced equidistantly along the length of the wire 802. In some other implementations, the spacing of the sensors 804 may vary along the length of the wire 802. For example, the sensors 804 can be spaced closer together in a distal portion of the wire 802 and can be spaced farther apart in a proximal portion of the wire 802.

The sensors 804 may implement any suitable sensing technology that can be used to determine their positions in a coordinate system. In some implementations, each sensor 804 may be an EM sensor that can be tracked in relation to an EM field (such as produced by the EM field generator 180 of FIGS. 1 and 3–5). For example, each sensor 804 may produce sensor data indicating its position in a given coordinate frame (such as the EM sensor space). In some implementations, the sensor data produced by the sensors 804 may be one example of the sensor data 606 of FIG. 6. More specifically, with reference to FIG. 6, a centroid 806 of the sensors 804 may be designated the target location 608. For example, the centroid 806 can be calculated by detecting the positions of each of the sensors 804 and determining the center (or average) of the sensor positions.

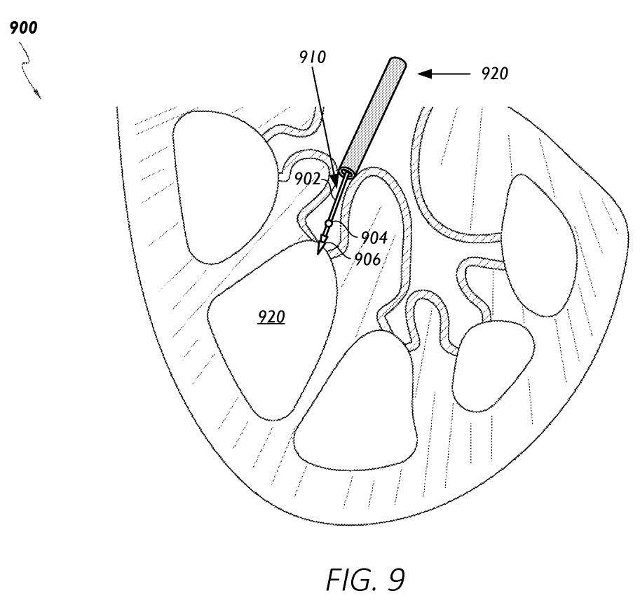

FIG. 9 shows another example positioning of a working channel instrument 910 for designating a target for percutaneous access in an anatomy 900, according to some implementations. In the example of FIG. 9, the anatomy 900 is depicted as a kidney. However, the working channel instrument 910 can be used to designate a target location in other types of anatomies using the same or similar techniques as described with reference to FIG. 9.

In the example of FIG. 9, the working channel instrument 910 includes a shaft 902 having a barbed tip 906 and a sensor 904 disposed on the length of the shaft 902 at a fixed distance from the barbed tip 906. The sensor 904 can be coupled or attached to the shaft 902 using any suitable means (including adhesives, laser welding, or other mechanical means). The shaft 902 can have any shape or geometry suitable for insertion through the working channel of an endoscope 920. More specifically, the shaft 902 can be formed from any semi-rigid material that can transfer forces axially along the length of the shaft 902 (such as from an instrument handle to the barbed tip 906) so that the barb 906 can be inserted at least partially into the anatomy 900 (such as by latching or hooking into a surface of the anatomy 900).

As shown in FIG. 9, the endoscope 920 is positioned proximate to a target anatomical feature 920 (such as a papilla) through which a percutaneous access needle is to be inserted. In some implementations, the endoscope 920 may be one example of the scope 120 of FIGS. 1–5. The shaft 902 extends beyond the distal opening of the endoscope 920 so that the barbed tip 906 is at least partially inserted into the papilla 920. More specifically, as shown in FIG. 9, the barb 906 can latch or hook into the papilla 920 to anchor the sensor 904 within an anatomical volume (such as a calyx). In this way, the sensor 904 may track any movements of the anatomy 900 without requiring constant pressure to be applied via the working channel instrument 910.

The sensor 904 may implement any suitable sensing technology that can be used to determine its position in a coordinate system. In some implementations, the sensor 904 may be an EM sensor that can be tracked in relation to an EM field (such as produced by the EM field generator 180 of FIGS. 1 and 3–5). For example, the sensor 904 may produce sensor data indicating its position in a given coordinate frame (such as the EM sensor space). In some implementations, the sensor data produced by the sensor 904 may be one example of the sensor data 606 of FIG. 6. More specifically, with reference to FIG. 6, the position of the sensor 904 while the barb 906 is latched to the papilla 920 may be designated the target location 608.

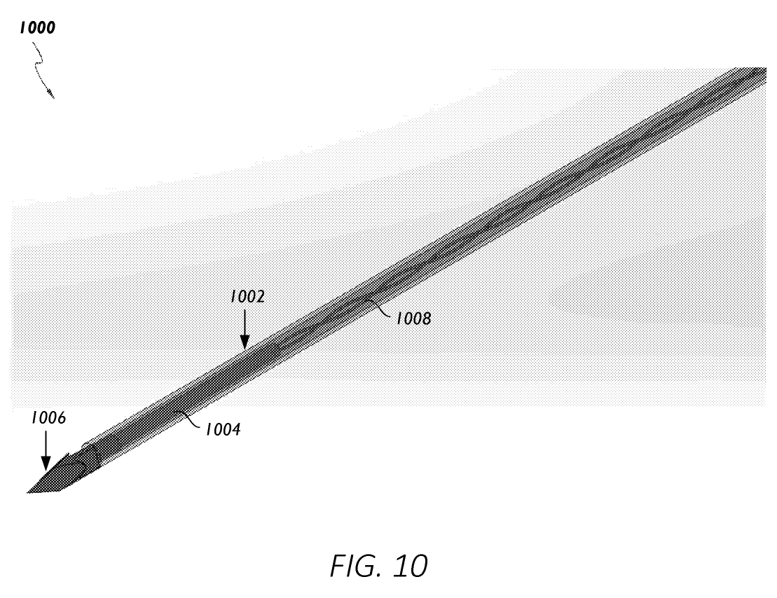

FIG. 10 shows an example working channel instrument 1000, according to some implementations. In some implementations, the working channel instrument 1000 may be one example of the working channel instrument 910 of FIG. 9. More specifically, the working channel instrument 1000 includes an elongate shaft 1002 having a barbed tip 1006 and a sensor 1004 disposed on the length of the shaft 1002 at a fixed distance from the barbed tip 1006. In the example of FIG. 10, the shaft 1002 is depicted as a cylindrical tube that houses the sensor 1004. More specifically, the shaft 1002 has an inner diameter or lumen that shields the sensor 1004 and wires 1008 (or conductors) carrying sensor data or electrical signals to and/or from the sensor 1004. As described with reference to FIG. 9, the barb 1006 is designed to latch or hook into an anatomical surface (such as a papilla) to anchor the sensor 1004 at a given distance relative to the anatomical surface (such as within a calyx).

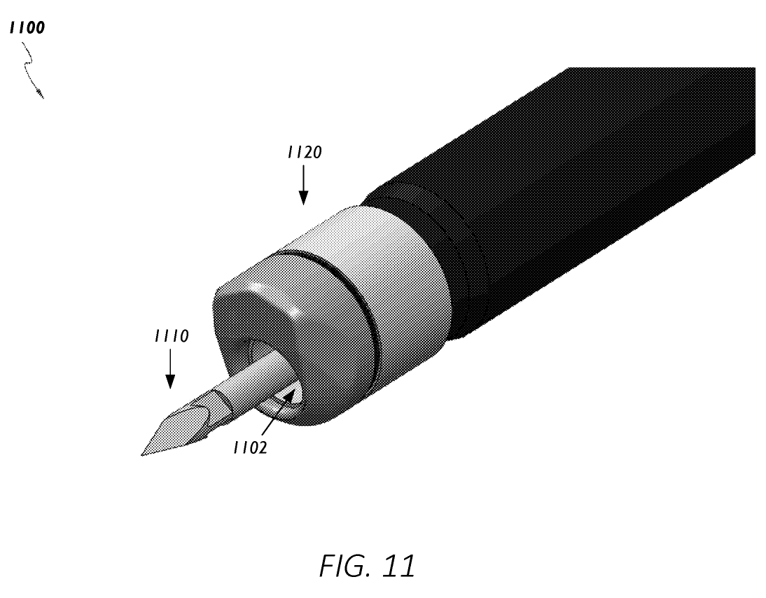

FIG. 11 shows an example instrument assembly 1100 that includes a working channel instrument 1110 and an endoscope 1120, according to some implementations. In some implementations, the endoscope 1120 may be one example of any of the endoscopes 720, 820, or 930 of FIGS. 7–9, respectively. As shown in FIG. 11, the working channel instrument 1110 can be inserted through a working channel 1102 (or lumen) of the endoscope 1120. In the example of FIG. 11, the working channel instrument 1110 is depicted as a shaft having a barbed tip (such as any of the working channel instruments 910 or 1000 of FIGS. 9 and 10, respectively). However, any working channel instrument can be inserted through the working channel 1102 of the endoscope 1120 (including the working channel instruments 710 or 810 of FIGS. 7 and 8, respectively). As described with reference to FIGS. 7–9, the working channel instrument 1110 can extend past the distal opening of the endoscope 1120 to contact one or more portions of an anatomy (such a calyx and/or a papilla).

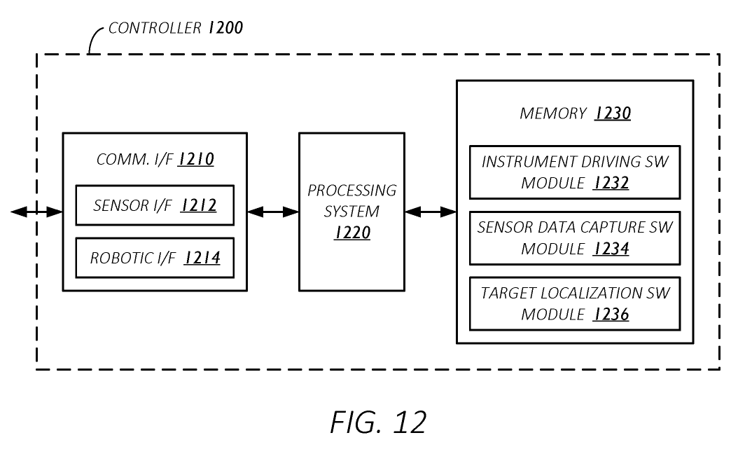

FIG. 12 shows a block diagram of an example controller 1200 for a medical system, according to some implementations. In some implementations, the controller 1200 may be one example of the target localization system 600 of FIG. 6 or any of the control circuitry 251 or 211 of FIG. 2. More specifically, the controller 1200 is configured to determine or designate a target location within an anatomy based, at least in part, on sensor data received from one or more position sensors disposed on a working channel instrument.

The controller 1200 includes a communication interface (I/F) 1210, a processing system 1220, and a memory 1230. The communication interface 1210 is configured to communicate with one or more components of the medical system. More specifically, the communication interface 1210 includes a sensor interface (I/F) 1212 for communicating with one or more position sensors (such as any of the sensors 704, 802–806, or 904 of FIGS. 7–9) and a robotic interface (I/F) 1214 for communicating with a robotic system (such as the robotic system 110 of FIGS. 1 and 2).

The memory 1230 may include a non-transitory computer-readable medium (including one or more nonvolatile memory elements, such as EPROM, EEPROM, Flash memory, or a hard drive, among other examples) that may store the following software (SW) modules: an instrument driving SW module 1232 to drive a first medical instrument within an anatomy, where the first medical instrument includes a working channel; a sensor data capture SW module 1234 to capture sensor data from one or more sensors disposed on a distal portion of a second medical instrument that is inserted through the working channel of the first medical instrument, where the sensor data indicates positions of the one or more sensors within the anatomy; and a target localization SW module 1236 to determine a target location within the anatomy for percutaneous access based at least in part on the sensor data.

The processing system 1220 may include any suitable one or more processors capable of executing scripts or instructions of one or more software programs stored in the controller 1200 (such as in the memory 1230). For example, the processing system 1220 may execute the instrument driving SW module 1232 to drive a first medical instrument within an anatomy, where the first medical instrument includes a working channel. The processing system 1220 also may execute the sensor data capture SW module 1234 to capture sensor data from one or more sensors disposed on a distal portion of a second medical instrument that is inserted through the working channel of the first medical instrument, where the sensor data indicates positions of the one or more sensors within the anatomy. Further, the processing system 1220 may execute the target localization SW module 1236 to determine a target location within the anatomy for percutaneous access based at least in part on the sensor data.

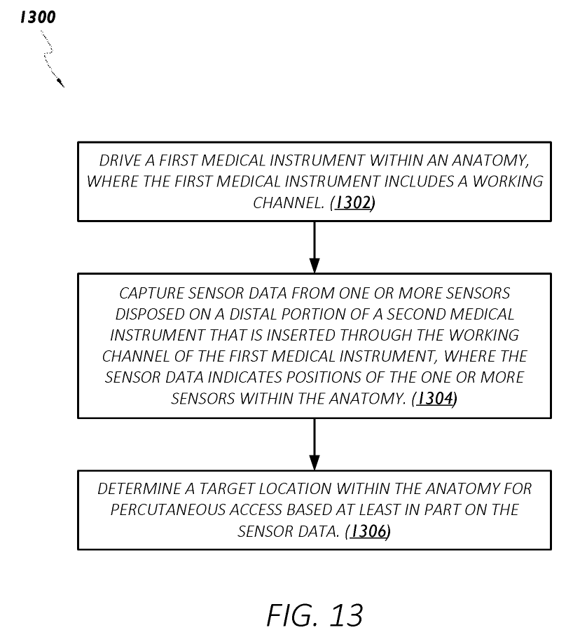

FIG. 13 shows an illustrative flowchart depicting an example target localization operation 1300, according to some implementations. In some implementations, the example operation 1300 may be performed by a controller for a medical system, such as the controller 1200 of FIG. 12.

The controller drives a first medical instrument within an anatomy, where the first medical instrument includes a working channel (1302). The controller also captures sensor data from one or more sensors disposed on a distal portion of a second medical instrument that is inserted through the working channel of the first medical instrument, where the sensor data indicates positions of the one or more sensors within the anatomy (1304). In some implementations, the controller may receive user input indicating that the distal portion of the second medical instrument is in contact with the anatomy, where the sensor data is captured responsive to receiving the user input. The controller further determines a target location within the anatomy for percutaneous access based at least in part on the sensor data (1306).

In some aspects, the first medical instrument may include an endoscope. In some implementations, the one or more sensors may include one or more EM sensors and the sensor data may indicate positions of the one or more EM sensors relative to an EM field. In some implementations, the anatomy may include a kidney and the target location may be associated with a calyx of the kidney.

In some aspects, the second medical instrument may include a flexible wire. In some implementations, the one or more sensors may include a sensor disposed on a distal tip of the flexible wire and the target location may represent the position of the sensor disposed on the distal tip of the flexible wire.

In some aspects, the flexible wire may be configured to coil or deform in response to contacting the anatomy. In some implementations, the one or more sensors may include a plurality of sensors disposed along a length of the flexible wire and the target location may represent a centroid of the positions of the plurality of sensors.

In some other aspects, the second medical instrument may include a shaft having a barbed tip that is configured to penetrate the anatomy. In some implementations, the one or more sensors may include a sensor disposed on the shaft at a fixed distance from the barbed tip and the target location may represent the position of the sensor disposed on the shaft.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method performed by a medical system, comprising:
driving a first medical instrument within an anatomy, the first medical instrument including a working channel;
capturing sensor data from one or more sensors disposed on a distal portion of a second medical instrument that is inserted through the working channel of the first medical instrument, the sensor data indicating positions of the one or more sensors within the anatomy; and
determining a target location within the anatomy for percutaneous access based at least in part on the sensor data.

2. The method of claim 1, further comprising:
receiving user input indicating that the distal portion of the second medical instrument is in contact with the anatomy, the sensor data captured responsive to receiving the user input.

3. The method of claim 1, wherein the anatomy comprises a kidney and the target location is associated with a calyx of the kidney.

4. The method of claim 1, wherein the one or more sensors include one or more electromagnetic (EM) sensors and the sensor data indicates the positions of the one or more EM sensors relative to an EM field.

5. The method of claim 1, wherein the first medical instrument comprises an endoscope.

6. The method of claim 1, wherein the second medical instrument comprises a flexible wire.

7. The method of claim 6, wherein the one or more sensors includes a sensor disposed on a distal tip of the flexible wire and the target location represents the position of the sensor disposed on the distal tip of the flexible wire.

8. The method of claim 6, wherein the flexible wire is configured to coil or deform in response to contacting the anatomy.

9. The method of claim 8, wherein the one or more sensors include a plurality of sensors disposed along a length of the flexible wire and the target location represents a centroid of the positions of the plurality of sensors.

10. The method of claim 1, wherein the second medical instrument comprises a shaft having a barbed tip that is configured to penetrate the anatomy.

11. The method of claim 10, wherein the one or more sensors include a sensor disposed on the shaft at a fixed distance from the barbed tip and the target location represents the position of the sensor disposed on the shaft.

12. A medical instrument, comprising:
an elongate member configured to be inserted through a working channel of an endoscope, the elongate member having a distal portion configured to contact an anatomy; and
one or more sensors disposed on the distal portion of the elongate member, the one or more sensors configured to produce sensor data indicating positions of the one or more sensors within the anatomy.

13. The medical instrument of claim 12, wherein the second medical instrument comprises a flexible wire.

14. The medical instrument of claim 13, wherein the one or more sensors includes a sensor disposed on a distal tip of the flexible wire and the target location represents the position of the sensor disposed on the distal tip of the flexible wire.

15. The medical instrument of claim 13, wherein the flexible wire is configured to coil or deform in response to contacting the anatomy.

16. The medical instrument of claim 15, wherein the one or more sensors include a plurality of sensors disposed along a length of the flexible wire and the target location represents a centroid of the positions of the plurality of sensors.

17. The medical instrument of claim 12, wherein the second medical instrument comprises a shaft having a barbed tip that is configured to penetrate the anatomy.

18. The medical instrument of claim 17, wherein the one or more sensors include a sensor disposed on the shaft at a fixed distance from the barbed tip and the target location represents the position of the sensor disposed on the shaft.

19. A controller for a medical system comprising:
a processing system; and
a memory storing instructions that, when executed by the processing system, cause the medical system to:
drive a first medical instrument within an anatomy, the first medical instrument including a working channel;
capture sensor data from one or more sensors disposed on a distal portion of a second medical instrument that is inserted through the working channel of the first medical instrument, the sensor data indicating positions of the one or more sensors within the anatomy; and
determine a target location within the anatomy for percutaneous access based at least in part on the sensor data.

20. The controller of claim 19, wherein execution of the instructions further causes the medical system to:
receive user input indicating that the distal portion of the second medical instrument is in contact with the anatomy, the sensor data captured responsive to receiving the user input.
